(19) United States
(12) Patent Application Publication
(10) Pub. No.: US 2016/0157887 A1
(43) Pub. Date: Jun. 9, 2016

(54) Apparatus For Generating Needle Insertion Path For Interventional Robot

(71) Applicants: Hong Ho KIM, Seoul (KR); Dong Gi WOO, Gunpo-si (KR); Yong Yeob CHA, Seoul (KR); Jun Woo PARK, Yongin-si (KR)

(72) Inventors: Hong Ho KIM, Seoul (KR); Dong Gi WOO, Gunpo-si (KR); Yong Yeob CHA, Seoul (KR); Jun Woo PARK, Yongin-si (KR)

(73) Assignee: Hyundai Heavy Industries Co. Ltd., Ulsan (KR)

(21) Appl. No.: 14/945,080

(22) Filed: Nov. 18, 2015

(30) Foreign Application Priority Data
Dec. 8, 2014 (KR) 10-2014-0174797

(51) Int. Cl. A61B 17/34; A61B 5/00; A61B 5/06; A61B 5/08; A61B 6/03; A61B 6/12

(52) U.S. Cl. CPC A61B 17/3403; A61B 19/2203; A61B 19/50; A61B 6/032; A61B 6/12; A61B 5/064; A61B 5/08; A61B 5/746; A61B 5/742

(57) ABSTRACT

Disclosed is a apparatus for generating a needle insertion path for an interventional robot, which provides a needle insertion path for intervention by inserting a needle. The apparatus includes a patient-side optical tool located on a patient for showing a position of an intervention target part among parts of the patient, a robot-side optical tool located on an interventional robot for showing a position of the interventional robot, and a needle path calculator configured to track positions of the patient-side optical tool and the robot-side optical tool to perform spatial registration on a local coordinate system of the patient-side optical tool based on the position of the patient-side optical tool with respect to a robot base coordinate system based on the position of the robot-side optical tool, and calculate a needle insertion path with respect to robot base coordinate system, based on a result of the spatial registration.

### CROSS-REFERENCE TO RELATED APPLICATIONS

This application claims the benefit of the Korean Patent Application No. 10-2014-0174797 filed on Dec. 8, 2014, which is hereby incorporated by reference as if fully set forth herein.

### BACKGROUND

1. Field of the Invention

The present invention relates to an apparatus for generating a needle insertion path for an interventional robot, in which a needle is inserted into a diseased part of a patient and biopsy or treatment is performed, and more particularly, to an apparatus for generating a needle insertion path for an interventional robot, which controls a robot according to a needle insertion position and a needle insertion path planned by an operator and monitors a respiration of a patient.

2. Discussion of the Related Art

Generally, intervention is technology that inserts a medical instrument into a human body and performs an interventional procedure while observing the inside of the human body through an imaging apparatus. Intervention is medical technology which is used for a surgical procedure and medical procedure such as tissue biopsy, dilation, medicine injection, etc.

As a type of intervention, a needle insertion type intervention uses a needle as a medical instrument. In the needle insertion type intervention, a needle is inserted into a human body, and an interventional procedure is performed. The needle insertion type intervention is applied to most intervention fields used as a method of approaching a lesion when installing various stents or a sheath, in addition to fields such as tissue biopsy of a chest, an abdomen, or various organ lesions and fields such as radio frequency, alcohol, coagulation, or brachytherapy for a lesion part.

In the needle insertion type intervention, an operator directly brings a medical needle in contact with a desired body part or a lesion to be treated while looking an image, obtained from a computed tomography (CT) apparatus or a magnetic resonance imaging (MRI) apparatus which is used in department of radiology, in a medical procedure and performs diagnosis or treatment. Recently, a method where an operator manipulates a manipulation unit of a needle insertion type interventional robot (a needle insertion robot) is used as disclosed in Korean Patent Publication No. 10-2014-0056772 (May 12, 2014).

In such a needle insertion type interventional robot system, since a needle insertion path planned by an operator is defined with respect to coordinate system of a diagnostic apparatus or an image by using a diagnostic image, target coordinates need to be converted into data with respect to a robot base coordinate system in order for a robot to be controlled to a target position. Such conversion technology is referred to as spatial registration.

However, in a related art of needle insertion type interventional robot system, a robot is installed and fixed where its position and orientation, relative to an image coordinate system, are already known. For this reason, suitability for a clinical environment is low, and it is necessary to perform calibration and replacement of components, repeatedly due to mechanical wear.

Moreover, in a related art of needle insertion type interventional robot system, since a patient is basically fixed by a compression instrument in order for the patient not to move and an operator inevitably checks the patient with eyes, fatigues of the patient and the operator are high, and its accuracy is not guaranteed. Therefore, a separate monitoring apparatus needs to be introduced for monitoring a respiration of a patient, and an operator should determine when a needle has to be inserted while looking at a monitoring situation. For this reason, an interventional process is very inconvenient and cumbersome.

### SUMMARY

Accordingly, the present invention is directed to provide an apparatus for generating a needle insertion path for an interventional robot that substantially obviates one or more problems due to limitations and disadvantages of the related art.

An aspect of the present invention is directed to provide an apparatus for generating a needle insertion path for an interventional robot, in which an optical tool is attached to each of a patient and an interventional robot, and a needle insertion path based on a robot base coordinate system is provided through spatial registration to which an optical tracking system is applied.

Another aspect of the present invention is directed to provide an apparatus for generating a needle insertion path for an interventional robot, which simultaneously monitors a movement and a respiration of a patient.

Additional advantages and features of the invention will be set forth in part in the description which follows and in part will become apparent to those having ordinary skill in the art upon examination of the following or may be learned from practice of the invention. The objectives and other advantages of the invention may be realized and attained by the structure particularly pointed out in the written description and claims hereof as well as the appended drawings.

To achieve these and other advantages and in accordance with the purpose of the invention, as embodied and broadly described herein, there is provided an apparatus for generating a needle insertion path for an interventional robot, which provides a needle insertion path for intervention, including: a patient-side optical tool located on a patient for showing a position of an intervention target inside patient; a robot-side optical tool located on an interventional robot for showing a position of the interventional robot; and a needle path calculator configured to track positions of the patient-side optical tool and the robot-side optical tool to perform spatial registration on a local coordinate system of the patient-side optical tool based on the position of the patient-side optical tool with respect to a robot base coordinate system based on the position of the robot-side optical tool, and calculate a needle insertion path with respect to a robot base coordinate system, based on a result of the spatial registration.

It is to be understood that both the foregoing general description and the following detailed description of the present invention are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this application, illustrate embodiments of the invention and together with the description serve to explain the principle of the invention. In the drawings:

FIG. 1 is a diagram illustrating a whole configuration of an apparatus for generating a needle insertion path for an interventional robot according to an embodiment of the present invention;

FIG. 2 is a conceptual diagram for describing the three-dimensional (3D) position tracking principle of an optical tool, in the apparatus for generating a needle insertion path for an interventional robot according to an embodiment of the present invention;

FIG. 3 is a diagram illustrating a concept for calculating positions and orientations of a robot-side optical tool and a patient-side optical tool, in the apparatus for generating a needle insertion path for an interventional robot according to an embodiment of the present invention;

FIG. 4 is a diagram illustrating a concept of a needle insertion path based on a robot base coordinate system, in the apparatus for generating a needle insertion path for an interventional robot according to an embodiment of the present invention; and

FIG. 5 is a diagram illustrating a function of a transformation matrix T.

### DETAILED DESCRIPTION OF THE INVENTION

Reference will now be made in detail to the exemplary embodiments of the present invention, examples of which are illustrated in the accompanying drawings. Wherever possible, the same reference numbers will be used throughout the drawings to refer to the same or like parts.

Hereinafter, details for disclosing an apparatus for generating a needle insertion path for an interventional robot according to an embodiment of the present invention will be described in detail with reference to the accompanying drawings.

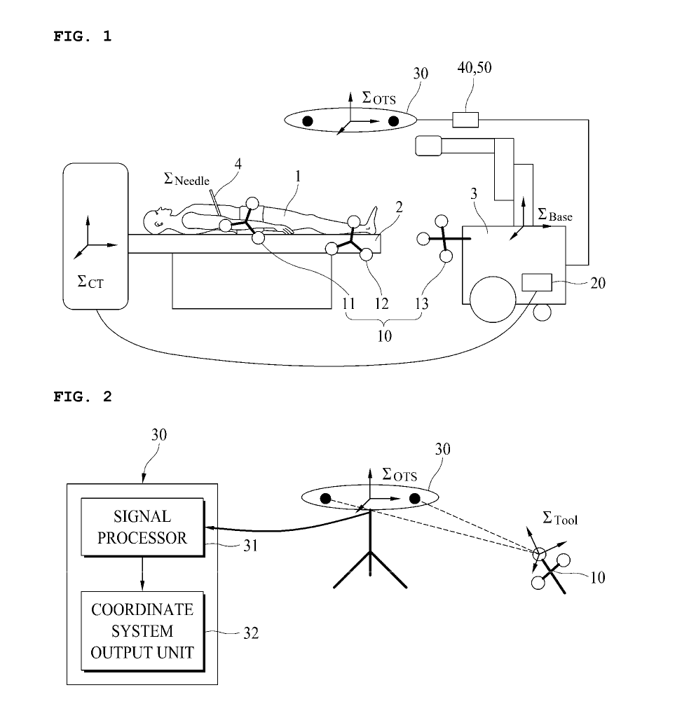

FIG. 1 is a diagram illustrating a whole configuration of an apparatus for generating a needle insertion path for an interventional robot according to an embodiment of the present invention. Referring to FIG. 1, the apparatus for generating a needle insertion path for an interventional robot according to an embodiment of the present invention may include: an optical tool 10 that displays a diseased part of a patient 1, a position of a CT bed 2, and a position of a robot 3; a CT image acquisition part 20 that receive CT scan images of the diseased part and the optical tool 10 attached to the patient 1; a position tracker 30 that tracks a robot-side optical tool and a patient-side optical tool with a stereo infrared camera; a needle path calculator 40 that performs spatial registration of the robot-side optical tool and the patient-side optical tool to calculate a needle insertion path, based on a robot base coordinate system; and a monitoring part 50 that monitors a movement and a respiration of the patient 1 for which intervention is being performed.

According to an embodiment of the present invention, a total of three reference coordinate systems (camera coordinate system “Σ_{OTS}”, robot base coordinate system “Σ_{Base}” that is the basis for robot control, and CT coordinate system “Σ_{CT}” for CT image) are introduced.

The optical tool 10 may include a patient-side optical tool 11 attached near a needle insertion area of the patient 1, a bed-side optical tool 12 attached to one side of the CT bed, and a robot-side optical tool 13 attached to a position having a fixed relative position and orientation with respect to the robot base coordinate system “Σ_{Base}”.

The optical tool 10 may include three or four bars having a branch form which are provided in different directions with respect to the center point. A highly retro-reflective ball marker may be provided on an end of each of the bars, and as illustrated in FIG. 2, the stereo infrared camera which includes a signal processor 31 and a coordinate system output unit 32 may track the ball marker attached to the optical tool 11.

That is, relative positions of the ball markers with respect to the camera coordinate system “Σ_{OTS}” may be measured by the stereo infrared camera in real time.

Relative 3D positions of the ball markers with respect to the camera coordinate system “Σ_{OTS}” may be measured by a triangulation technique in real time. In such an optical tracking system, by measuring 3D positions of three ball markers, a plane in a space may be determined, and orientations of the ball markers may be measured.

Therefore, an optical tool 10 may be attached to a target object which is to be measured and may measure a relative position or movement, and a reference coordinate system may be tracked by pre-calibration of relationship between a pose of target and a reference coordinate system.

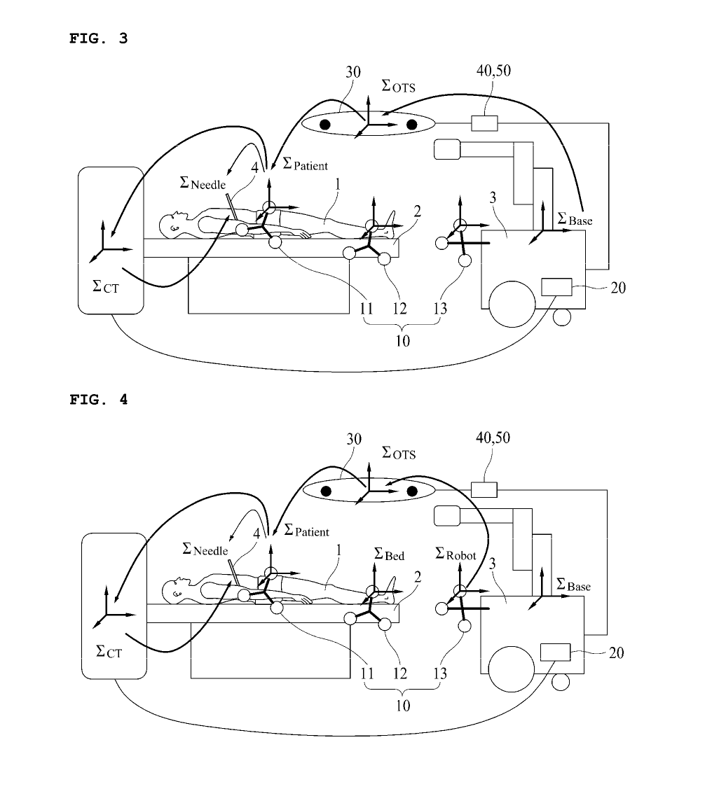

As described above, with the patient-side optical tool 11 “Σₚₐₜᵢₑₙₜ” among the optical tool 10 according to an embodiment of the present invention being attached to a position near a needle insertion point of the patient 1 as illustrated in FIG. 3, the CT image acquisition part 20 may receive image data by CT scan, and center points of ball markers with respect to the CT image coordinate system “Σ_{CT}” may be extracted from the received image data.

When the center points of the ball markers and information of a needle insertion path are generated, the needle insertion path based on CT image coordinate system “Σ_{CT}” may be transformed into data based on a local coordinate system of the patient-side optical tool 11.

A Relationship between the robot-side optical tool 13 and robot base coordinate system “Σ_{Base}” may be identified by pre-calibration. The pre-calibration may be that a separate optical tool is attached to a robot arm, and the robot arm is driven in a state of matching a reference axis of a robot base coordinate system. Then relative position and orientation of robot base coordinate system with respect to a local coordinate system of a robot-side optical tool is identified.

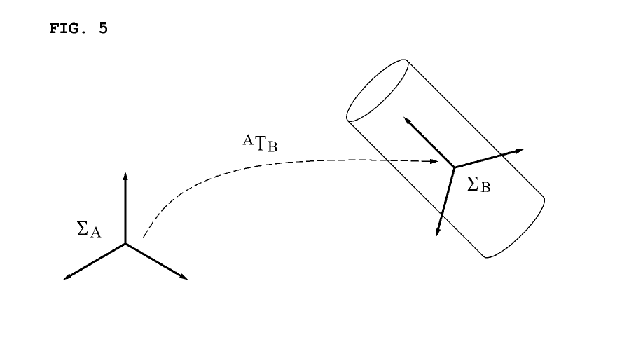

In the present embodiment, as illustrated in FIG. 4, the needle path calculator 40 may simultaneously track the robot-side optical tool 13 and the patient-side optical tool 11 by using the optical tracking system to calculate a needle insertion path based on the robot base coordinate system. This may be explained by the following Equation (1):

^{Base}T_{Needle}=^{Base}T_{Robot} ^{Robot}T_{OTS} ^{OTS}T_{Patient} ^{Patient}T_{CT} ^{CT}T_{Needle} (1)

An interventional robot can be driven to a position of the needle insertion path, accurately.

As described above, the optical tracking system of the position tracker 30 may simultaneously track the robot-side optical tool 13 and the patient-side optical tool 11, and thus, the needle path calculator 40 may calculate needle insertion path data, based on the robot base coordinate system, whereby spatial registration may be performed.

The spatial registration enables interventional robot to be installed at a medical procedure place with low limitations in terms of position.

Moreover, the apparatus for generating a needle insertion path for an interventional robot according to an embodiment of the present invention may include a bed-side optical tool 12 where a ball marker is attached to one side of CT bed 2, and may measure a relative movement of the local coordinate system “Σₚₐₜᵢₑₙₜ” of the patient-side optical tool 11 with respect to the local coordinate system “Σ_{Bed}” of the bed-side optical tool 12, thereby checking a movement and a respiration state of the patient 1. According to the present embodiment, a movement of the patient 1 may be defined as the following Equation (2), a respiration of the patient 1 may be defined by measuring an absolute movement of the patient 1 as the following Equation (3):

^{Bed}T_{Patient}=^{Bed}T_{OTS} ^{OTS}T_{Patient} (2)

^{OTS}T_{Patient} (3)

Here, a matrix T of Equations (1) to (3) may denote that a relative position and orientation between two coordinate systems in a 3D space are expressed as a 3D homogeneous transformation matrix of the following Equation (4):

$$T = [ \begin{matrix}{r}_{11} & {r}_{12} & {r}_{13} & {t}_{x} \\ {r}_{21} & {r}_{22} & {r}_{23} & {t}_{y} \\ {r}_{31} & {r}_{32} & {r}_{33} & {t}_{z} \\ 0 & 0 & 0 & 1\end{matrix} ] \qquad ( 4 )$$

where a 3×3 region including r₁₁ to r₃₃ denotes a change of orientation (i.e., 3D rotation) between two coordinates, and tₓ, t_{y} and t_{z} denote distances between origins of two coordinate systems.

All elements of the matrix T has no unit because the transformation matrix T depends on a unit of a coordinate value for 3D rotation or movement.

FIG. 5 is a diagram illustrating a function of the transformation matrix T.

Referring to FIG. 5, a relative position and orientation of a B coordinate system “Σ_{B}” with respect to an A coordinate system “Σ_{A}” may be expressed as ^{A}T_{B}, and a relative position and orientation with respect to another coordinate system may be obtained by sequentially performing a multiplication operation on the 3D homogeneous transformation matrix according to the chain rule.

When a movement of patient-side optical tool 11 is outside an allowable value, the monitoring part 50 according to an embodiment of the present invention may output a warning sound, display a warning message on a screen, or generate a signal for stopping driving of the interventional robot.

The monitoring part 50 according to an embodiment of the present invention may track in real time a relative movement of a patient-side optical tool with respect to an optical tool attached to CT bed, thereby monitoring a movement and a respiration of a patient. Accordingly, a device for fixing a patient is not needed, and it is not required for an operator to check the patient with eyes. Also, an accuracy of intervention and a stability of a medical procedure are secured.

To summarize the above-described technology disclosed in the present specification, the technology disclosed in the present specification relates to the apparatus for generating a needle insertion path for an interventional robot, which provides a needle insertion path for intervention by inserting a needle. The apparatus for generating a needle insertion path for an interventional robot according to an embodiment of the present invention may include: a patient-side optical tool 11 that is located on a patient for indicating a position of an intervention target part among parts of the patient; a robot-side optical tool 13 that is located on an interventional robot for indicating a position of the interventional robot; and a needle path calculator 40 that tracks positions of the patient-side optical tool 11 and the robot-side optical tool 13 to perform spatial registration on a local coordinate system of the patient-side optical tool 11 based on the position of the patient-side optical tool 11 with respect to a robot base coordinate system based on the position of the robot-side optical tool 13, and calculates a needle insertion path, based on a result of the spatial registration.

Moreover, the apparatus for generating a needle insertion path for an interventional robot according to an embodiment of the present invention may further include a CT image acquisition part 20 that receives image data including the patient-side optical tool 11 and the intervention target part.

Moreover, the image data may be generated by performing a CT scan of the patient-side optical tool 11 and the intervention target part simultaneously.

Moreover, the apparatus for generating a needle insertion path for an interventional robot may further include a position tracker 30 that calculates a local coordinate system of the patient-side optical tool 11, based on the CT scan image and tracks the position of the patient-side optical tool 11, by the optical tracking system.

Moreover, the robot-side optical tool 13 may be attached to robot arm or robot base of an interventional robot 3. The position tracker 30 may calculate a position to which the robot-side optical tool 13 is attached and a relative position and orientation of a robot base coordinate system, based on the optical tracking system.

Moreover, the position of the robot-side optical tool 13 may be tracked based on a stereo camera.

Moreover, the patient-side optical tool 11 or the robot-side optical tool 13 may include three or four bars having a branch form which are provided in different directions with respect to a center point. A highly retro-reflective ball marker may be provided on an end of each of the bars.

Moreover, the apparatus for generating a needle insertion path for an interventional robot may further include a bed-side optical tool 12 that is attached to one side of a CT bed. Also, the apparatus for generating a needle insertion path for an interventional robot may further include a monitoring part 50 that is coupled to the position tracker 30 and checks the position of the patient-side optical tool 11 with respect to a local coordinate system of the bed-side optical tool 12 to check a movement and a respiration state of the patient.

Moreover, when a movement of the patient is outside an allowable value, the monitoring part 50 may output a warning sound, display a warning message on a screen, or generate a signal for stopping driving of the interventional robot.

As described above, in the apparatus for generating a needle insertion path for an interventional robot according to the embodiments of the present invention, the interventional robot may be installed with a low limitation, and a needle insertion path may be determined based on the robot base coordinate system, thereby enhancing an accuracy of intervention.

Moreover, since a movement and a respiration of a patient are monitored in real time through optical position tracking, it is possible to issue a warning to an operator or stop driving of a system, and thus, intervention is safely and accurately performed.

It will be apparent to those skilled in the art that various modifications and variations can be made in the present invention without departing from the spirit or scope of the inventions. Thus, it is intended that the present invention covers the modifications and variations of this invention provided they come within the scope of the appended claims and their equivalents.

What is claimed is:

1. An apparatus for generating a needle insertion path for an interventional robot, which provides a needle insertion path for intervention by inserting a needle, the apparatus comprising:
a patient-side optical tool located on a patient for showing a position of an intervention target part among parts of the patient;
a robot-side optical tool located on an interventional robot for showing a position of the interventional robot; and
a needle path calculator configured to track positions of the patient-side optical tool and the robot-side optical tool to perform spatial registration on a local coordinate system of the patient-side optical tool based on the position of the patient-side optical tool with respect to a robot base coordinate system based on the position of the robot-side optical tool, and calculate a needle insertion path with respect to robot base coordinate system, based on a result of the spatial registration.

2. The apparatus of claim 1, further comprising: a computed tomography (CT) image acquisition part configured to receive CT image including the patient-side optical tool and the intervention target part.

3. The apparatus of claim 2, wherein the CT image is generated by performing a CT scan of the patient-side optical tool and the intervention target part.

4. The apparatus of claim 2, further comprising: a position tracker configured to calculate a local coordinate system of the patient-side optical tool, based on the CT image and track the position of the patient-side optical tool, based on an optical tracking system.

5. The apparatus of claim 4, wherein
the robot-side optical tool is attached to an robot arm or a robot base of the interventional robot, and
the position tracker calculates a position of robot base coordinate system by tracking the robot-side optical tool based on the optical tracking system.

6. The apparatus of claim 5, wherein the position of the robot-side optical tool is tracked based on a stereo camera.

7. The apparatus of claim 1, wherein
the patient-side optical tool and the robot-side optical tool comprises three or four bars having a branch form which are provided in different directions with respect to a center point, and
a highly retro-reflective ball marker is provided on an end of each of the bars.

8. The apparatus of claim 1, further comprising:
a bed-side optical tool located on one side of a medical procedure bed; and
a monitoring part coupled to the position tracker to check the position of the patient-side optical tool based on a local coordinate system of the bed-side optical tool to check a movement and a respiration state of the patient.

9. The apparatus of claim 8, wherein when a movement of the patient is outside an allowable value, the monitoring part outputs a warning sound, displays a warning message on a screen, or generates a signal for stopping driving of the interventional robot.

10. The apparatus of claim 4, further comprising:
a bed-side optical tool located on one side of a medical procedure bed; and
a monitoring part coupled to the position tracker to check the position of the patient-side optical tool based on a local coordinate system of the bed-side optical tool to check a movement and a respiration state of the patient.

11. The apparatus of claim 10, wherein when a movement of the patient is outside an allowable value, the monitoring part outputs a warning sound, displays a warning message on a screen, or generates a signal for stopping driving of the interventional robot.
